(19) United States
(12) Patent Application Publication
(10) Pub. No.: US 2002/0137704 A1
(43) Pub. Date: Sep. 26, 2002

(54) Methods of inhibiting host-cell mediated viral replication

(76) Inventors: David M. Markovitz, Ann Arbor, MI (US); Neil Faulkner, Ypsilanti, MI (US); Brian Lane, Ann Arbor, MI (US)

Correspondence Address: HARNESS, DICKEY & PIERCE, P.L.C., P.O. BOX 828, BLOOMFIELD HILLS, MI, 48303, US

(21) Appl. No.: 09/878,541

(22) Filed: Jun. 11, 2001

Related U.S. Application Data
(60) Provisional application No. 60/211,134, filed on Jun. 13, 2000.

(51) Int. Cl. A61K 48/00; A61K 31/35

(52) U.S. Cl. 514/44; 514/454

(57) ABSTRACT

Methods are provided for inhibiting or suppressing viral replication in an infected host cell. More specifically, methods are provided for inhibiting or suppressing viral replication in an infected host cell by administering compounds that inhibit cellular protein phosphatase 2A. Such methods are advantageous for treating viral infections such human immunodefeciency virus infections.

### SPONSORSHIP

Work on this invention was sponsored in part by National Institutes of Health Grant Al36685. The Government may have certain rights in the invention.

### FIELD OF THE INVENTION

The present invention relates generally to methods of inhibiting host cell mediated viral replication and, more particularly, to methods of inhibiting cellular enzymes in the host cell required for viral replication.

### BACKGROUND OF THE INVENTION

Medical therapy for infection with the human immunodeficiency virus type 1 (HIV-1) has improved over the last several years. However, many patients still fail to respond to the available pharmacologic agents, due to drug resistance and other factors. In addition, the antivirals currently available are often not well tolerated by patients.

The currently available drugs for treating HIV-1 infection either attack the reverse transcriptase or protease enzymes of the virus. Such compounds have the problem of rapidly developing drug resistance due to rapid transformation of the infecting viral genome. However, despite the problem of rapid drug resistance such drugs have been used to treat HIV seropositive individuals to help prevent the progression of the infection into the clinical symptoms of acquired immunodeficiency syndrome (AIDS). Such drugs are also used to obtain regression of AIDS.

Viral replication in a host cell requires not only viral reverse transcriptase and proteases, but also enzymes of the host cell. Inhibition of the host cells enzymes involved in viral replication would decrease the development of drug resistance by rapidly mutating viruses. U.S. Pat. No. 5,985,926 discloses methods for inhibiting viral replication by inhibiting cellular signaling through a specific phospho lipid-based cellular signaling and signal amplification pathway. U.S. Pat. No. 5,750,394 discloses compounds that inhibit the interaction of nucleoprotein of influenza A virus with the NP-1 protein of the host cell, thereby inhibiting viral replication.

One attractive cellular target is controlling protein phosphorylation in the host cell through the kinases and phosphatases involved. Many eukaryotic cell functions, including signal transduction, cell adhesion, gene transcription, RNA splicing, apoptosis and cell proliferation are controlled by protein phosphorylation. Protein phosphorylation is in turn, regulated by the dynamic relationship between kinases and phosphatases.

Agents such as phorbol esters, TNF-α, and CD3 crosslinking antibodies activate kinase cascades culminating in the induction of HIV long terminal repeat (LTR) transcriptional activity. Although the roles of various kinases activated by these agents, such as protein kinase C (PKC), IκB kinase (IKK), and mitogen activated kinase (MAPK), in regulation of the HIV-1 LTR has been well documented, little is known about the actions of specific phosphatases on LTR activity. Phosphoprotein phosphatase 2A (PP2A) constitutes a significant portion of the serine/threonine-specific protein phosphatase activity found within many cells. Schonthal, A. H. Front. Biosci. 3:D1262-73 (1998). The PP2A holoenzyme is comprised of three subunits denoted A (65 kDa regulatory subunit), B (55 kDa regulatory subunit), and C (37 kDa catalytic subunit). Schonthal, A. H. Front. Biosci. 3:D1262-73 (1998); Orgis, E. et al., Oncogene 15(8):911-7. These subunits associate to form an AC dimer (the core enzyme) or an ABC trimer (holoenzme), each of which has different substrate specificities. Okadaic acid (OKA), a marine sponge toxin which specifically inhibits protein phosphatases-1 and -2A (PP1 and PP2A), has been reported to activate the HIV-1 LTR. Riekmann, P. et al., Biochem. Biophys. Res. Commun. 187(1):51-7 (1992); Li, M. et al., Biochem. Biophys. Res. Commun. 202(2):1023-30 (1994); Thevenin, C. et al., New Biologist 2(9): 793-800 (1990); Vlach, J. et al. Virology 208(2):753-61 (1995). Evidence has suggested that the mechanism through which OKA acts involves blocking the dephosphorylation of IκB by PP2A, thereby accelerating its phosphorylation and degradation. This releases the IκB-tethered NF-κB which undergoes translocation from the cytosol to the nucleus, where it binds its cognate enhancer sequences in the HIV-1 LTR. OKA has also been reported to enhance the phosphorylation of Sp1, leading to an increase in activation of an HIV-1 LTR reporter construct devoid of NF-κB response elements. Vlach, J. et al. Virology 208(2):753-61 (1995). OKA-induced Sp1-mediated transcription is further enhanced by the presence of Tat. Vlach, J. et al. Virology 208(2):753-61 (1995); Chun, R. F. et al., J. Virol. 72(4):2615-29 (1998). These studies with OKA suggest the involvement of one or more phosphatases (PP2A or PP1) in regulating transcriptional activation of the HIV-1 LTR. More recently, studies with direct evidence for the involvement of PP2A in HIV-1 LTR regulation have been published. Increasing the ratio of PP2A core enzyme to holoenzyme by using an N-terminal mutant of the A subunit of PP2A inhibited tat stimulated HIV-1 transcription and virus production. Ruediger, R. et al., Virology 238(2):432-43 (1997).

PP2A is known to modulate the activity of a number of signal-transducing factors some of which regulate HIV LTR activity including mitogen-activated protein (MAP) kinases, NF-κB, AP-1, Sp1, raf-1, Ca²⁺- calmodulin-dependent kinase IV (CaMKIV) and PKC. Millward, T. A. et al., Trends Biochem. Sci. 24(5):186-91 (1999). Direct regulation of PP2A activity by viruses plays a significant role in mediating viral replication and cell transformation. Both simian virus 40 (SV40) and polyoma viruses encode antigens, small-t and middle-T respectively, that form complexes with PP2A core enzyme. Yang, S. I. et al., Mol. Cell Biol. 11(4):1988-95 (1991). These complexes while not transforming on their own, facilitate or enhance the cellular transformation processes caused by these viruses. The importance of PP2A to cell survival and growth is reflected in the fact that the intracellular levels of the catalytic domain are post-translationally regulated in a number of cell lines. Baharians, Z. et al., J. Biol. Chem. 273(30):19019-24 (1998). Autoregulation of PP2A levels have made it very difficult to obtain stable overexpression of the catalytic domain of the protein and observe an increase in enzymatic activity. Recently, expression vectors driven by a CMV promoter have been employed to overexpress the C subunit of PP2A with a concomitant increase in intracellular PP2A phosphatase activity.

It would thus be desirable to provide a method of controlling viral replication in a host cell targeting the host cell's enzymes. It would also be desirable to provide methods for treating a patient with a viral infection. Preferably such methods will inhibit or suppress viral replication resulting in the prevention, delay or abatement of the symptoms associated with a viral infection.

### SUMMARY OF THE INVENTION

Methods for suppressing viral replication in a viral infected host cell are provided. The methods comprise inhibiting cellular protein phosphatase 2A (PP2A) to suppress viral replication. PP2A actually stimulates viral replication and inhibition of PP2A thus suppresses replication.

Also provided are methods of treating viral infections, comprising administering to a patient a therapeutically effective amount of a composition comprising at least one inhibitor of PP2A. Such methods are particularly useful in treating patients that test seropositive for HIV.

Additional objects, advantages, and features of the present invention will become apparent from the following description and appended claims, taken in conjunction with the accompanying drawings.

### BRIEF DESCRIPTION OF THE DRAWINGS

The various advantages of the present invention will become apparent to one skilled in the art by reading the following specification and subjoined claims and by referencing the following drawings in which:

FIG. 1A is a graph showing PP2A activation of the HIV-2 promoter;

FIG. 1B is a graph showing that PP2A activation of the HIV-2 promoter is mediated by the pets site;

FIG. 2A is a schematic showing the HIV-2 CAT reporters used for transfections;

FIG. 2B is a graph showing PP2A activation of the HIV-2 promoters in the presence and absence of TPA stimulation;

FIG. 2C is a graph showing PP2A activation of the HIV-2 promoters in the absence of TPA stimulation;

FIG. 2D is a graph showing Tat-2 activation of the HIV-2 promoters;

FIG. 3 is a graph showing PP2A stimulation of HIV-1 replication;

FIG. 4 is a graph showing inhibition of HIV-1 replication by okadaic acid; and

FIG. 5 is a graph showing inhibition of HIV-1 replication by fostreicin.

### DETAILED DESCRIPTION OF THE PREFERRED EMBODIMENTS

Methods for suppressing viral replication in infected host cells are provided. In one embodiment, a method of the present invention comprises treating the infected host cell with an inhibitor of protein phosphatase 2A (PP2A). In a preferred embodiment, the host cell is infected with human immunodeficiency virus (HIV). The virus may be either HIV-1 or HIV-2, preferably HIV-1. In an alternate preferred embodiment, the host cell is either a monocyte or a T-cell.

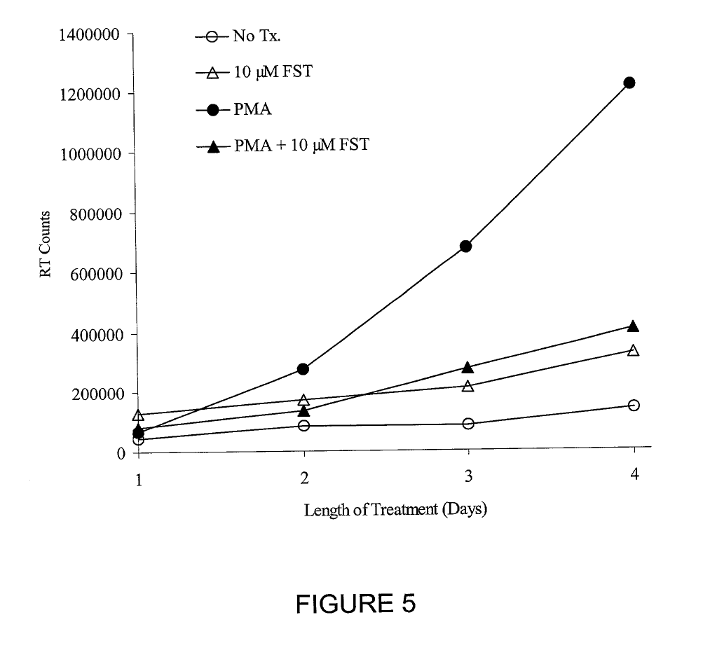

PP2A has been found to stimulate HIV-1 replication in infected host cells (FIG. 5). This result was surprising and unexpected in light of the prior art which taught that cellular PP2A suppressed HIV-1 replication. For example, okadaic acid inhibition of PP2A has been shown to activate the HIV-1 LTR and thus, stimulate replication. Riekmann, P. et al., Biochem. Biophys. Res. Commun. 187(1):51-7 (1992); Li, M. et al., Biochem. Biophys. Res. Commun. 202(2):1023-30 (1994); Thevenin, C. et al., New Biologist 2(9): 793-800 (1990); Vlach, J. et al. Virology 208(2):753-61 (1995). In the present invention, PP2A inhibitors such as okadaic acid actually suppress HIV-1 replication. See Specific Example 4. Stimulation of HIV-1 replication of HIV-1 was further unexpected because the HIV-1 promoter lacks the pets region which is involved in PP2A stimulation of HIV-2 promoter driven transcription, which is directly related to replication. See Specific Example 2.

The PP2A inhibitors used in the methods of the present invention are any compounds known to inhibit cellular PP2A. Such compounds may be synthetic compounds, natural products, proteins or peptides, or nucleic acid molecules. Non-limiting examples of PP2A inhibitors are okadaic acid, fostreicin, and calyculin A. Fostreicin is currently being used as topoisomerase II inhibitor for the treatment of cancer. deJong, R. S. et al., Br. J. Cancer 79(5-6):882-7 (1999). U.S. Pat. No. 6,040,323 also discloses PP2A inhibitors that can be used in the methods of the present invention. Inhibiting PP2A activity involves any method that will reduce, suppress or eliminate PP2A cellular activity. This may involve binding of an inhibitor directly to the enzyme. Alternatively, intracellular antibodies against PP2A can be used to inhibit PP2A activity. Intracellular antibodies are expressed by a gene that is inserted into the cell for this particular purpose. Once expressed in the cell, the antibodies will bind to PP2A, preventing the enzyme from carrying out catalysis.

In one aspect of this embodiment, the method of the present invention comprises treating a virus-infected host cell with a molecule that inhibits or suppresses the expression of cellular PP2A. For example, the infected host cell may be treated with antisense nucleic acid molecules specific for the gene encoding PP2A. Inhibition or suppression of expression of PP2A will then suppress or inhibit viral replication.

In one aspect of this embodiment, the method of the present invention comprises gene therapy whereby a gene encoding for a dominant mutant PP2A having significantly decreased activity or no activity is inserted into the infected host cell. Production of the dominant mutant will suppress expression of wild-type PP2A.

In a further embodiment, a method is provided for treating viral infections comprising administering to a patient diagnosed with a viral infection, a therapeutically effective amount of an inhibitor of cellular PP2A. Treatment of the viral infected cells in the viral infection with an inhibitor of PP2A results in suppression of viral replication and either suppress or slow down development of the disease. Treatment can also provide a decrease of any symptoms of the viral infection that are present. The methods of the present invention are particularly useful for treating patients suffering from HIV infection to prevent the development of full blown AIDS. The methods of the present invention are also useful for treating a patient having AIDS.

As used herein, the terms “therapeutically effective amount” and a “therapeutically effective duration” preferably mean the total amount of each active component of the pharmaceutical composition and a duration of treatment that is sufficient to show a meaningful patient benefit, i.e., treatment, healing, prevention or amelioration of the relevant medical condition, or an increase in rate of treatment, healing, prevention or amelioration of such conditions without undue adverse physiological effects or side effects. The term “therapeutically effective amount,” when applied to an individual active ingredient, administered alone, the term refers to that ingredient alone. When applied to a combination, the term refers to combined amounts of the active ingredients, e.g., PP2A inhibitors, that result in the therapeutic effect, whether administered in combination, serially or simultaneously.

An inhibitor of cellular PP2A of the present invention may thus be used in a pharmaceutical composition when combined with a pharmaceutically acceptable carrier. Such a composition may also contain (in addition to inhibitors of cellular PP2A and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s).

In practicing the method of treatment or use of the present invention, a therapeutically effective amount of inhibitors of cellular PP2A of the present invention are administered to a patient having a condition to be treated, e.g., HIV infection. Inhibitors of cellular PP2A of the present invention may be administered in accordance with the method of the invention either alone or in combination, including in combination with other conventional therapies. For example, inhibitors of cellular PP2A can be used as part of a multidrug regime.

Administration of inhibitors of cellular PP2A of the present invention used in the pharmaceutical composition or to practice the method of the present invention can be carried out in a variety of conventional ways, such as oral ingestion, inhalation, topical application or cutaneous, subcutaneous, intraperitoneal, parenteral or intravenous injection.

When a therapeutically effective amount of an inhibitor of cellular PP2A of the present invention is administered orally, the inhibitor of cellular PP2A of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% PP2A inhibitors of the present invention, and preferably from about 25 to 90% inhibitors of cellular PP2A of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical compositions may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of inhibitors of cellular PP2A of the present invention and preferably from about 1 to 50% inhibitors of cellular PP2A of the present invention.

When a therapeutically effective amount of an inhibitor of cellular PP2A of the present invention is administered by intravenous, cutaneous or subcutaneous injection, the inhibitor will be in the form of a pyrogen-free, parenterally acceptable aqueous solution. The preparation of such parenterally acceptable PP2A inhibitor solutions, having due regard to pH, isotonicity, stability, and the like, is within the skill in the art. A preferred pharmaceutical composition for intravenous, cutaneous, or subcutaneous injection should contain, in addition to inhibitors of cellular PP2A, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The amount of inhibitors of cellular PP2A of the present invention in the pharmaceutical composition of the present invention will depend upon the nature and severity of the condition being treated, and on the nature of prior treatments which the patient has undergone. Ultimately, the attending physician will decide the amount of inhibitors of cellular PP2A of the present invention with which to treat each individual patient. Initially, the attending physician will administer low doses of inhibitors of cellular PP2A and observe the patient's response. Larger doses of inhibitors of cellular PP2A may be administered until the optimal therapeutic effect is obtained for the patient, and at that point the dosage is not increased further.

It must be noted that, as used in this specification and the amended claims, the singular forms “a”, “an”, and “the” include plural referents unless context clearly dictates. Thus, for example, a reference to “an inhibitor of PPZA” includes mixtures of such inhibitors.

The foregoing may be better understood in connection with the following examples, which are presented for purposes of illustration and not by way of limitation.

### SPECIFIC EXAMPLE 1

### PP2A Activation of the HIV-2 Promoter

I. Methods

Cell Culture. Human U937 monocytic cells were maintained in RPMI 1640 medium containing 10% fetal bovine serum (GibcoBRL), penicillin (50 units/ml), streptomycin (50 μg/ml), and L-glutamine (2 mM).

Phosphatase Inhibition. U937 cells were treated with 5 to 50 nM okadaic acid (OKA) (Calbiochem) only or pretreated for 30-45 minutes with OKA prior to stimulation with 32 nM TPA (Sigma).

Transfections. 5 μg of CAT reporter construct, pCMV5 PP2Ac, and the empty pCMV5 vector were combined to yield 25 μg total DNA. U937 undergoing logarithmic growth were harvested at 5×10⁵ cells/ml and resuspended at 25×10⁶ cells/ml in RPMI 1640 media. 10⁷ cells (0.4 ml) were aliquoted to 0.4 cm electroporation cuvettes (Invitrogen) and incubated with the appropriate DNA mixture for 5 min at room temperature. The cells were then electroporated using Invitrogen's Electroporator II at a setting of 330 V, 1000 μF, and infinite resistance with an input voltage of approximately 325 V. Following electroporation, the cells from each cotransfection were evenly split (5×10⁷ cells/plate) and placed in separate tissue culture plates in 5 ml of RPMI1640 media+10% FBS. After a period of 12-16 hrs, one group of cells was left untreated while the other received 32 nM TPA for 24 hrs. The cells were then harvested, washed with PBS, and lysed in 0.25 M Tris-HCl (pH 7.5). Protein levels of the lysates were determined by BioRad's colorimetric protein assay (BioRad).

Chloramphenicol Acetyl Transferase (CAT) Assay. For CAT assays, 5-30 μl of cell lysate was combined with 20 μl of 100 mM Tris-HCl (pH 7.5). The final volume was increased to 50 μl with lysis buffer, if necessary, and the mixture incubated for 15 min at 65° C. to inactivate endogenous acetyltransferases. The lysates were then transferred to 7 ml scintillation vials and 200 μl CAT reaction mixture (125 mM Tris-HCl, 1.25 mM chloramphenicol, 0.1 μCi ³H-acetyl CoA) added. Five ml of Econofluor (Fisher Scientific) scintillation fluid was overlaid and the reaction incubated for 15-90 min before counting. Background and total counts were determined by mixing 200 μl CAT reaction mixture with 5 ml of Econfluor or Scintiverse (Fisher Scientific) respectively. CAT activity was background subtracted and normalized for total amount of protein used.

II. Results

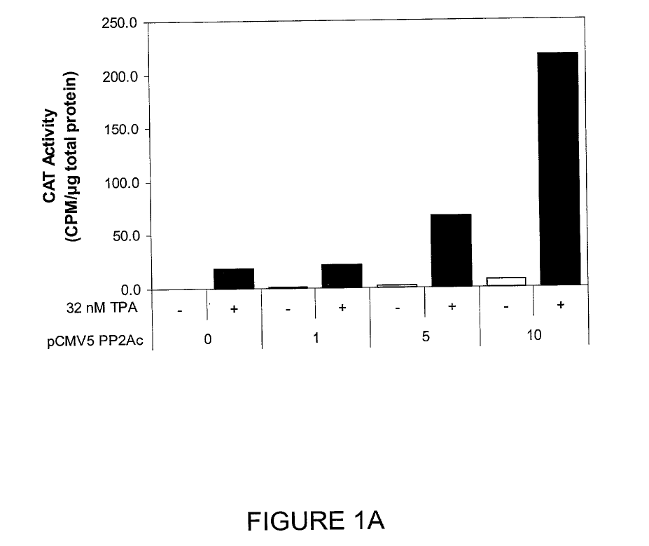

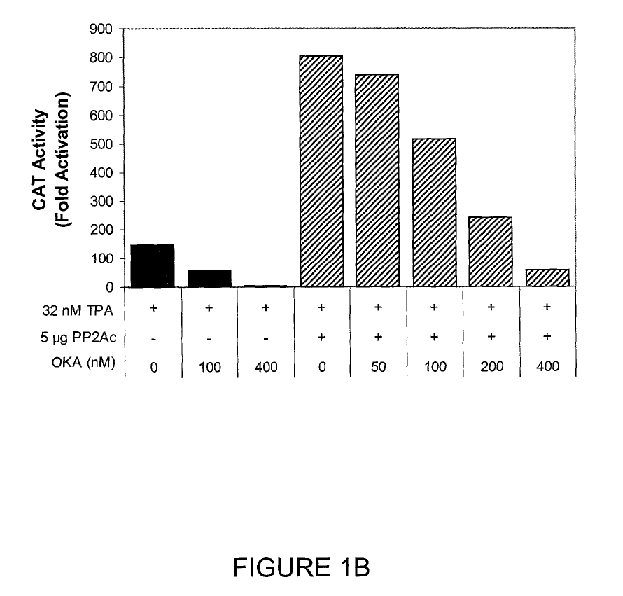

To test the possibility that PP2A is involved in the activation of the HIV-2 LTR and that PP2A may act on the HIV-2 LTR through the pets site, U937 cells were transfected with an HIV-2 enhancer CAT reporter alone or with pCMV5 PP2Ac, a plasmid encoding the catalytic domain of PP2A. After transfection, cells from each condition were split into an untreated control sample, to assess the baseline effects of PP2A, or a sample that received 32 nM 12-O-tetradecanoyl phorbol-13-acetate (TPA) for 24 hrs. CAT assays were performed and the results are shown in FIG. 1A. Increasing levels of ectopic PP2Ac from 1 to 10 mg enhanced both the baseline and TPA-induced activity of the HIV-2 CAT reporter significantly over no treatment or TPA alone, respectively. To further assure that the effects observed were indeed due to increased levels or activity of PP2A activity, cells were transfected with HIV-2 CAT alone or in conjunction with 5 μg of pCMV5 PP2Ac. The cells were pretreated with 50, 100, 200, or 400 nM OKA for 30 min prior to TPA treatment or treated with OKA alone for 18 hrs. CAT assays were performed and the values normalized relative to the baseline activity in the absence of TPA and/or ectopic PP2Ac. As shown in FIG. 1 B, TPA in the absence of ectopic PP2Ac resulted in a 148 fold increase in CAT activity above baseline. Ectopic PP2Ac coupled with TPA treatment resulted an average increase of 750 fold over the baseline activity. OKA alone enhanced CAT reporter activity in a dose dependent fashion with the maximal activity (a 150 fold increase) occurring at 200 nM. This is most likely due to activation of NF-κB and Sp1 by OKA as has been previously reported by Rieckmann, P. et al., Biochem. Biophys. Res. Commun. 187(1):51-7 (1992), Thevenin, C. et al., New Biologist 2(9):793-800 (1990), Vlach, J. et al., Virology 208(2):753-61 (1995) and Chun, R. F. et al., J. Virol. 72(4):2615-29 (1998). The addition of OKA to TPA-treated U937 cells resulted in a decrease in CAT activity, suggesting that OKA was antagonizing the effects of TPA (FIG. 1B). This antagonizing action of OKA on TPA activation of the HIV-2 CAT reporter has also been observed for the HIV-1 enhancer in Jurkats, a T-lymphocytic cell line. Thevenin, C. et al., New Biologist 2(9):793-800 (1990). Additionally, the synergy exhibited between PP2Ac and TPA in activating HIV-2 enhancer CAT reporter gene was also significantly reduced in a dose dependent manner by OKA. These data further support and extend the observations that PP2A phosphatase activity appears to be important in the signaling cascade culminating at the HIV-2 LTR.

### SPECIFIC EXAMPLE 2

### Role of the Pets Site in PP2A Activation of the HIV-2 Promoter

I. Methods

Cell Culture. Human U937 monocytic cells were maintained in RPMI 1640 medium containing 10% fetal bovine serum (GibcoBRL), penicillin (50 units/ml), streptomycin (50 μg/ml), and L-glutamine (2 mM).

Phosphatase Inhibition. U937 cells were treated with 5 to 50 nM okadaic acid (OKA) (Calbiochem) only or pretreated for 30-45 minutes with OKA prior to stimulation with 32 nM TPA (Sigma).

Transfections. 5 μg of CAT reporter construct, pCMV5 PP2Ac, and the empty pCMV5 vector were combined to yield 25 μg total DNA. U937 undergoing logarithmic growth were harvested at 5×10⁵ cells/ml and resuspended at 25×10⁶ cells/ml in RPMI 1640 media. 10⁷ cells (0.4 ml) were aliquoted to 0.4 cm electroporation cuvettes (Invitrogen) and incubated with the appropriate DNA mixture for 5 min at room temperature. The cells were then electroporated using Invitrogen's Electroporator II at a setting of 330 V, 1000 μF, and infinite resistance with an input voltage of approximately 325 V. Following electroporation, the cells from each cotransfection were evenly split (5×10⁷ cells/plate) and placed in separate tissue culture plates in 5 ml of RPMI1640 media+10% FBS. After a period of 12-16 hrs, one group of cells was left untreated while the other received 32 nM TPA for 24 hrs. The cells were then harvested, washed with PBS, and lysed in 0.25 M Tris-HCl (pH 7.5). Protein levels of the lysates were determined by BioRad's calorimetric protein assay (BioRad).

Chloramphenicol Acetyl Transferase (CAT) Assay. For CAT assays, 5-30 μl of cell lysate was combined with 20 μl of 100 mM Tris-HCl (pH 7.5). The final volume was increased to 50 μl with lysis buffer, if necessary, and the mixture incubated for 15 min at 65° C. to inactivate endogenous acetyltransferases. The lysates were then transferred to 7 ml scintillation vials and 200 μl CAT reaction mixture (125 mM Tris-HCl, 1.25 mM chloramphenicol, 0.1 μCi ³H-acetyl CoA) added. Five ml of Econofluor (Fisher Scientific) scintillation fluid was overlaid and the reaction incubated for 15-90 min before counting. Background and total counts were determined by mixing 200 μl CAT reaction mixture with 5 ml of Econfluor or Scintiverse (Fisher Scientific) respectively. CAT activity was background subtracted and normalized for total amount of protein used.

Results:

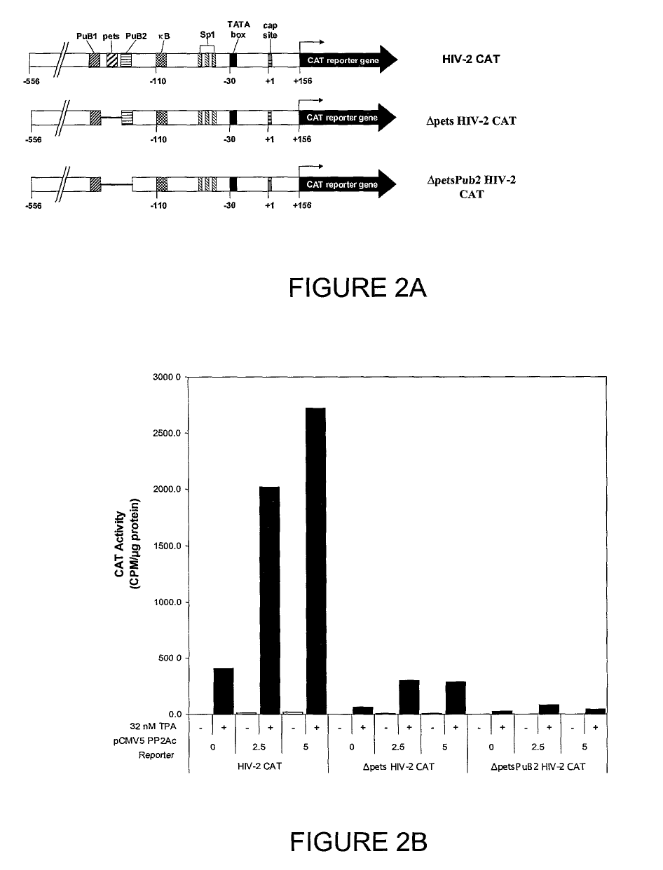

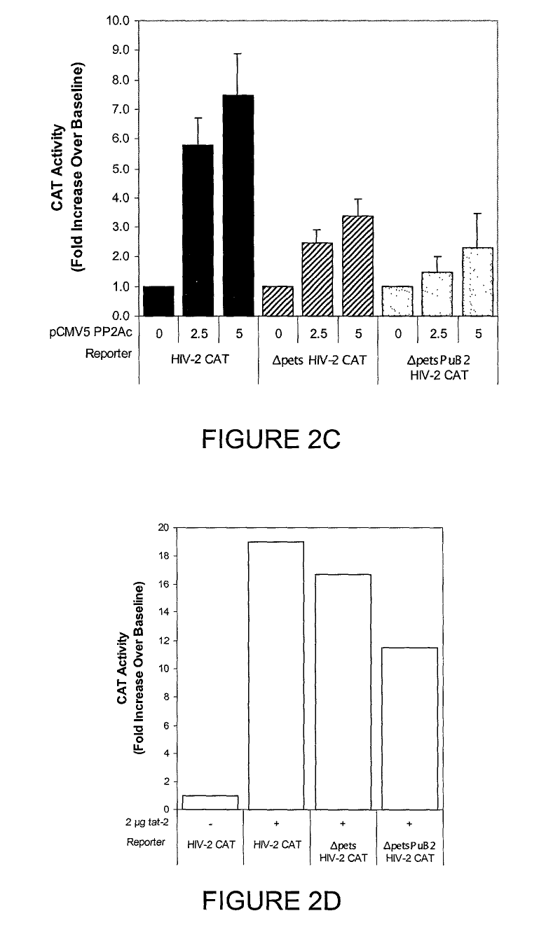

Since binding to the pets site of HIV-2 is modulated by phosphatase activity, it was sought to determine if the activation by PP2A and the synergy observed between PP2A and TPA is mediated by the pets response element. Mutant constructs lacking the pets site or both the pets and the adjacent PuB2 sites were used in these experiments (FIG. 2A). The PuB2 site has been shown to bind members of the Ets family of transcription factors, including Ets-1 and Elf-1. Markovitz, D. M. et al., J. Virol. 66(9):5479-84 (1992); Hannibal, M. C. et al., Blood 83(7):1839-46; Hilfinger, J. M. et al., J. Virol. 67(7):4448-53 (1993). Co-transfection of the mutant reporter constructs with increasing amounts of PP2Ac cDNA was performed and cells from each transfection condition split into two groups. One group was treated with 32 nM TPA for 24 hrs while the other went untreated. Mutation of the pets site led to a significant decrease in the TPA and PP2Ac synergy at the level of the HIV-2 LTR and a further decrease could be observed with mutation of the PuB2 site (FIG. 2B). Since PP2Ac increases the basal activity of the HIV-2 CAT reporter construct (FIG. 1A), the effect of mutating the pets or pets+Pub2 sites on the basal activity of the HIV-2 LTR in the presence of ectopic PP2Ac was also examined. Both sets of mutations resulted in a significant (P≦0.02) decrease in the basal activity of the HIV-2 CAT reporter in the presence of 2.5 or 5 μg of co-transfected pCMV5 PP2Ac (FIG. 2C). While not wishing to be bound by theory, the data in FIGS. 2B and 2C strongly support the involvement of the pets and PuB2 elements in the activation of the HIV-2 LTR seen with ectopic PP2Ac and TPA treatment of U937 cells. To address concerns that the deletions in the enhancer region of the CAT reporters had a non-specific adverse effect on the promoters, U937 cells were cotransfected with Tat-2 and the mutant constructs. CAT assays were performed 24 hrs after 32 nM TPA treatment or no treatment. As can be seen in FIG. 2D, Tat-2 in the absence of TPA leads to a significant increase in CAT activity over baseline for all the constructs. The Tat-2 mediated increase in CAT activity is further augmented by the addition of TPA. This demonstrates that the mutant reporters are still transcriptionally active and potentially highly inducible, consistent with previously published reports from our laboratory utilizing these reporters. Markovitz, D. M. et al., J. Virol. 66(9):5479-84 (1992); Hannibal, M. C. et al., Blood 83(7):1839-46; Hilfinger, J. M. et al., J. Virol. 67(7):4448-53 (1993).

### SPECIFIC EXAMPLE 3

### PP2A Stimulation of HIV-1 Replication

Methods: U937 monocytic cells were transfected with 2 μg of the infectious HIV-1 clone HXB2 and 2.5 μg of the catalytic domain of PP2A. The cells were treated with PMA (32 nM) 24 hours after transfection. Reverse Transcriptase (RT) activity was measured in supernatants collected each day after transfection. The data represent the mean of the RT activity present in triplicate wells an are representative of three independent experiments.

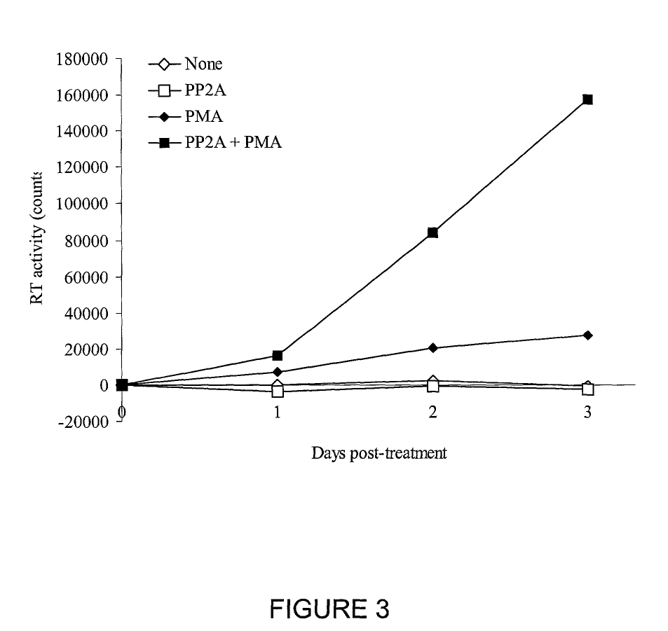

Results: PP2A, in concert with phorbol ester PMA, stimulates HIV-1 replication (closed squares, FIG. 3). PP2A alone showed no effect on viral replication over control (open squares and open diamonds, respectively, FIG. 3). The stimulation of HIV-1 replication in the presence of both PP2A and PMA was synergistic, with RT activity approximately 8-fold higher in cultures with both as compared to PMA alone (closed diamonds, FIG. 3).

### SPECIFIC EXAMPLE 4

### Inhibition of HIV-1 Replication by PP2A Inhibitors

Methods: U1 cells, a monocytic cell line chronically infected with HIV-1, were treated with okadaic acid at several concentrations for 1.5-5.5 hours prior to treatment with the phorbol ester PMA (32 nM). Supernatants were collected on days 1, 2, 3, 4 after infection to be analyzed for viral replication by reverse transcriptase (RT) assay. Peak replication was observed at 3 or 4 days after infection. Data shown are the mean of the RT values from 2 to 4 independent experiments for each point (±SEM).

U1 cells, a monocytic cell line chronically infected with HIV-1, were treated with fostreicin (FST) at several concentrations for 1.5 hours prior to treatment with the phorbol ester PMA (32 nM). Supernatants were collected on days 1, 2, 3, 4 after infection and analyzed for viral replication by reverse transcriptase (RT) assay. Data shown are the mean of the RT values (counts) from triplicate wells.

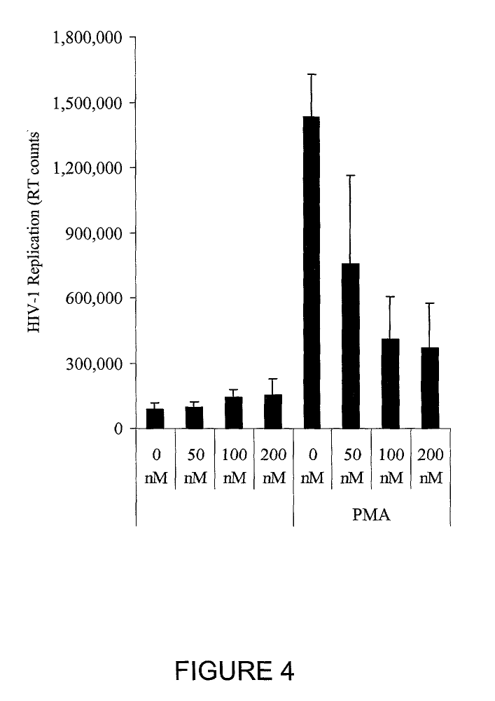

Results: The PP2A inhibitors, okadaic acid and FST, both inhibit PMA-stimulated HIV-1 replication (FIGS. 4 and 5). Okadaic acid inhibited replication in a concentration dependent manner from 50 nM to 200 nM (FIG. 4). There was no inhibition in the absence of PMA.

FST, at a concentration of 10 μM, totally reversed PMA stimulation of HIV-1 replication (filled triangles v. filled circles, FIG. 5). Cellular viability was minimally affected by treatment with FST. The cellular viability was >100% for 10 μM FST compared to no treatment and 76.3% for 10 μM FST and PMA compared with PMA alone.

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

All references cited herein including literature references and patents, are incorporated by reference as if fully set forth

We claim:

1. A method of inhibiting or suppressing viral replication in a host cell infected with a virus, the method comprising administering to the host cell a compound which inhibits cellular protein phosphatase 2A.

2. The method of claim 1, wherein the compound binds to protein phosphotase 2A.

3. The method of claim 2, wherein the compound is okadaic acid.

4. The method of claim 2, wherein the compound is fostriecin.

5. The method of claim 1, wherein the compound inhibits expression of active cellular protein phosphatase 2A.

6. The method of claim 5, wherein the compound is an antisense nucleic acid molecule.

7. The method of claim 5, wherein the compound is a vector comprising a gene encoding dominant mutant inactive form of protein phosphatase 2A.

8. The method of claim 1, wherein the host cell is a monocyte.

9. The method of claim 1, wherein the host cell is a T-cell.

10. The method of claim 1, wherein the virus is HIV.

11. A method for treating a patient having a viral infection, the method comprising administering to the patient a compound which inhibits cellular protein phosphatase 2A.

12. The method of claim 11, wherein the compound binds to cellular protein phosphatase 2A.

13. The method of claim 12, wherein the compound is okadaic acid.

14. The method of claim 12, wherein the compound is fostriecin.

15. The method of claim 11, wherein the compound inhibits expression of active cellular protein phosphatase 2A.

16. The method of claim 11, wherein the compound is an antisense nucleic acid molecule.

17. The method of claim 11, wherein the compound is a vector comprising a gene encoding dominant mutant inactive form of protein phosphatase 2A

18. The method of claim 11, wherein the compound inhibits protein phosphatase 2A in monocytes in the patient.

19. The method of claim 11, wherein the compound inhibits protein phosphatase 2A in T-cells in the patient.

20. The method of claim 11, wherein the viral infection is caused by HIV-1.

21. Use of a compound which inhibits cellular protein phosphatase 2A for the production of a medicament for administration to a patient having a viral infection.
